(19) United States
(12) Patent Application Publication
(10) Pub. No.: US 2010/0268003 A1
(43) Pub. Date: Oct. 21, 2010

(54) PROCESS FOR INCREASING PURITY OF SOLID BROMINATED FLAME RETARDANTS DURING PREPARATION

(75) Inventors: Saadat Hussain, Baton Rouge, LA (US); Arthur G. Mack, Prairieville, LA (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 12/745,951

(22) PCT Filed: Nov. 24, 2008
(86) PCT No.: PCT/US08/84546

§ 371 Date: Jun. 3, 2010

Related U.S. Application Data
(60) Provisional application No. 61/012,139, filed on Dec. 7, 2007.

(51) Int. Cl. C07C 17/00

(52) U.S. Cl. 570/201; 570/190

(57) ABSTRACT

Processes for preparing brominated aromatic flame retardant having a reduced content of occluded free bromine are described. The processes comprise subdividing, during bromination of an aromatic compound, solid particles that form in a bromination reaction mixture in which an excess of liquid bromine and a Lewis acid bromination catalyst are present, said subdividing taking place within the confines of said reaction mixture. Subdivision is effected by grinding, milling, or sonication.

### BACKGROUND

A considerable variety of brominated flame retardants are relatively high melting solids. During their production, especially when an excess of bromine is used, a portion of the bromine ends up in the product as occluded free bromine, a term which refers to that molecular bromine (Br₂) which is tightly held by the brominated flame retardant product so that ordinary washing techniques are insufficient to reduce its content within the product. Not only is this wasteful of bromine, but the presence of this occluded bromine in the product adversely affects its purity.

In U.S. Pat. No. 6,518,468, reference is made in column 8 to a method wherein a wet cake of a solid brominated flame retardant is submitted to a dry and grind technique, such as subjecting a flame retardant to dry grinding in a hammer mill to reduce the average particle size and to reduce the amount of occluded free bromine. As noted in that patent, at least the product described therein will still contain 700 to 1000 ppm occluded free bromine, a level which still exceeds the more acceptable and desired free bromine level of 150 to 200 ppm of occluded free bromine.

### BRIEF SUMMARY OF THIS INVENTION

This invention provides, inter alia, a new way of providing higher purity brominated flame retardant by materially reducing the amount of occluded free bromine in the brominated flame retardant product as it is being produced.

Thus, in one of its embodiments, this invention provides a process in which a higher purity brominated aromatic flame retardant is formed, which process comprises continuously breaking up or subdividing (e.g., by use of grinding or sonication) the product particles during bromination of the aromatic compound being brominated. In a more particular embodiment, this invention is applied to production of brominated aromatic flame retardants in which each aromatic ring is perbrominated in a process wherein an excess of liquid bromine is employed as the brominating agent and a Lewis acid bromination catalyst is used.

The above and other embodiments of this invention will be still further apparent from the ensuing description and appended claims.

### FURTHER DETAILED DESCRIPTION OF EMBODIMENTS OF THIS INVENTION

In one of its embodiments, this invention provides a process of preparing brominated aromatic flame retardant having a reduced content of occluded free bromine, which process comprises subdividing during bromination of an aromatic compound, solid particles that form in a bromination reaction mixture in which an excess of liquid bromine and a Lewis acid bromination catalyst are present, said subdividing taking place within the confines of said reaction mixture. While some forms of Lewis acid catalysts may be present as solids, their presence usually will not unduly interfere with the subdividing activity taking place within the body of the reaction mixture. However, it is preferable to charge aluminum bromide as the Lewis acid bromination catalyst as this is soluble in liquid bromine and thus does not provide additional solids in the reaction mixture.

Preferably, the processes of this invention are utilized when producing aromatic flame retardant compounds that are perbrominated or essentially perbrominated. By “essentially perbrominated” is meant that an average of no more than one unsubstituted brominatable ring position in the compound being brominated remains unbrominated.

This invention is applicable to the production of a wide variety of brominated aromatic flame retardant compounds that are produced by bromination of the corresponding unbrominated compound or corresponding partially brominated compound. Some non-limiting examples of such flame retardant compounds are pentabromotoluene, tetrabromophthalic anhydride, decabromodiphenyl oxide (a.k.a. decabromodiphenyl ether), decabromobiphenyl, tetradecabromodiphenoxy benzene, pentabromophenol, and decabromodiphenylethane. Preferred products of this invention are decabromodiphenyl oxide and decabromodiphenylethane. These are best produced by bromination of diphenyloxide or diphenylethane, respectively, in a sea of liquid bromine using a suitable Lewis acid bromination catalyst. As is well known in the art, the bromination of diphenylethane is conducted in the absence of light to minimize the possibility of aliphatic bromination. The amount of excess bromine used in a sea of bromine process can be varied widely, but should be sufficient to maintain about 10 moles of excess of bromine at all times. Typically, the reaction mixture will contain in the range of about 14 to about 25 moles of bromine per mole of aromatic compound being or to be brominated. It is possible to use more than 25 moles bromine per mole of aromatic compound in order to provide an even greater reserve of bromine to also serve as excess solvent for the reaction.

While various Lewis acid bromination catalysts can be used, iron-based catalysts such as subdivided iron (e.g., iron powder, iron filings, etc.), ferric chloride, ferric bromide, or mixtures of such materials are preferred. More preferred are aluminum-based catalysts such as metallic aluminum (e.g., in the form of aluminum foil, aluminum turnings, aluminum flakes, etc.), aluminum chloride, aluminum bromide, aluminum chlorodibromide, aluminum bromodichloride, or mixtures of such materials. During the initial stages of the bromination reaction, particles of iron or aluminum may exist as solids until they have reacted to be transformed into a soluble form of iron or aluminum halide. Thus, it is preferable to charge into the reaction mixture, a ferric halide or aluminum halide in which the halogen atoms are chlorine and/or bromine atoms. Typically, these materials are rapidly transformed into soluble forms in the reaction mixture.

Temperatures of the bromination reactions are usually relatively low, e.g., in the range of about 50 to about 65° C., and typically operating under superatmospheric pressure when necessary to keep bromine in the liquid state.

Pursuant to this invention, use can be made of various methods and equipment for effecting the breaking up of the solids as they form during the bromination reaction. For example, use can be made of grinding or milling apparatus disposed within the body of the reaction mixture. Such apparatus should be fabricated from corrosion-resistant materials of construction, a number of which are available as articles of commerce. As between grinding and milling apparatus, grinding apparatus is preferred as it tends to result in more uniform agitation of the reaction mixture.

One type of apparatus which can be used for effecting breakup of the brominated product as formed during the bromination is an appliance within the reaction vessel, which appliance is composed of a receptacle of hard, corrosion-resistant material having a bowl-shaped cavity in which the solids are ground with an internally or externally operated pestle device fabricated from the same or similar hard, corrosion-resistant material. The portions of such device within the reaction vessel can be fabricated from of suitably hard ceramic materials, such as porcelain, or suitably corrosion-resistant metal alloys. Nickel-tungsten alloys and iron-based chromium alloys serve as potential suitably corrosion-resistant metal alloy candidate materials for portions of the device within the reaction vessel.

Another type of apparatus which can be used for effecting breakup of the brominated product as formed during the bromination and within the confines of the reaction mixture is sonication apparatus, especially ultrasonication apparatus, which involves use of high frequency sound waves. Such apparatus can be in the form of a bath sonicator in which sonic energy from a transducer is transferred to the particles through the liquid phase of the reaction mixture (e.g., liquid bromine or an inert organic solvent or diluent containing liquid bromine), or in the form of one or more probe sonicators which are immersed in the reaction mixture and which transmit such sonic energy through the liquid phase of the reaction mixture. In practice, the sonication apparatus can be activated either continuously or intermittently during the bromination reaction, but at least should be activated as perbromination of the aromatic ring(s) is approached. The sonication apparatus should be encased in corrosion-resistant material such as glass or stainless steel, or both. The frequency and amplitude output characteristics of the sonication apparatus used will be dependant to some extent upon the identity of the flame retardant being formed and the composition of the bromination reaction mixture undergoing bromination. Use of sonication apparatus producing ultrasonic waves having a resident frequency from about 15 to about 100 kHz and an amplitude, when measured peak-to-peak, in the range of from about 10 to about 100 microns is recommended at least as a starting point for determining optimum sonication conditions for any given bromination reaction mixture to be processed pursuant to this invention.

In embodiments of this invention where the bromination is conducted with liquid bromine and catalyst in the presence of an inert organic solvent or diluent, the solvent or diluent is typically a halogenated solvent such as, for example, bromochloromethane, dichloromethane, 1,2-dichloroethane, 1,2-dibromoethane, or other suitable liquid aliphatic halohydrocarbons in which the halogen atoms in the molecule are bromine atoms, chlorine atoms, or a combination of both. Halocarbons such as carbon tetrachloride can also be used. Mixtures of two or more such solvents or diluents can be used, if desired.

In order to determine the composition and purity of the brominated product formed in a process of this invention, a gas chromatographic procedure is used. The gas chromatography is conducted on a Hewlett-Packard 5890 Series II gas chromatograph (or equivalent) equipped with a flame ionization detector, a cool on-column temperature and pressure programmable inlet, and temperature programming capability. The column is a 12 AQ HT5 capillary column, 12 meter, 0.15μ film thickness, 0.53 mm diameter, available from SGE, Inc., part number 054657. Conditions are: detector temperature 350° C.; inlet temperature 70° C.; heating at 125° C./min to 350° C. and holding at 350° C. until the end of the run; helium carrier gas at 10 ml/min.; inlet pressure 4.0 psi, increasing at 0.25 psi/min. to 9.0 psig and holding at 9.0 psi until the end of the run; oven temperature 60° C. with heating at 12° C./min. to 350° C. and holding for 10 min.; and injection mode of cool on-column. Samples are prepared by dissolving, with warming, 0.003 grams in 10 grams of dibromomethane and injection of 2 microliters of this solution. The integration of the peaks is carried out using Target Chromatography Analysis Software from Thru-Put Systems, Inc. However, other and commercially available software suitable for use in integrating the peaks of a chromatograph may be used. Thru-Put Systems, Inc. is currently owned by Thermo Lab Systems, whose address is 5750 Major Blvd., Suite 200, Orlando, Fla. 32819. The address of SGE, Incorporated is 2007 Kramer Lane, Austin, Tex. 78758. Results are reported as GC area percents.

Determination of the amount of occluded bromine in the final product involves use of a procedure involving several determinations. In brief, the procedure yielding a determination of occluded bromine in decabromodiphenylethane is as follows: The sample is dissolved in 1,2,4-trichlorobenzene to release the occluded bromine and bromide. The bromine is then reduced to bromide by the addition of an aqueous sodium sulfite solution. The bromide is extracted into the aqueous phase and determined by ion chromatography. The total of occluded bromine and bromide is calculated from this result. To determine the occluded bromine the same procedure is repeated without using sodium sulfite. The bromide from the free bromide in the sample and bromide formed from hydrolysis of occluded bromine is extracted into the aqueous phase and determined by ion chromatography. The occluded bromine content is estimated from this uncorrected ionic bromide result and the total free bromine and bromide result. About one half of the occluded bromine is converted to bromide at low bromine levels in accordance with the equation:

Br₂+H₂O=>HBr+HOBr

Therefore, the occluded bromine is estimated as follows:

ppm free bromine=2×(ppm total of free bromine and bromide−ppm of uncorrected ionic bromide).

The ionic bromine is estimated as follows:

ppm of ionic bromine=ppm total occluded bromine and bromide−ppm of occluded bromine.

In greater detail, the apparatus and procedure used to determine occluded bromine and/or ionic bromine (bromide) in decabromodiphenylethane is as follows:
A) The required equipment includes a Dionex DX-500 ion chromatograph or equivalent, equipped with a conductivity detector; a Dionex PeakNet chromatography data collection and processing system and a Dionex IonPac®AS11-HC column equipped with Dionex IonPac® AG11-HC guard column.
B) The ion chromatographic operating conditions involve (a) as eluent: EG40 KOH gradient, (b) flow-rate: 1.5 mL/min, (c) injection volume: 25 μL, (d) detector range: 200 μS, (e) suppressor: ASRS-Ultra 4 mm, (f) suppressor current: 100 mA, and (g) regenerant: Autosuppression recycle mode.
C) The EG40 operating conditions are as listed in the following table.

| Time | Condition | Concentration |
|---|---|---|
| −7.100 | Concentration = | 30.00 |
| −7.00 | Concentration = | 5.00 |
| −1.200 | Autosampler Closed | |
| 0.000 | ECD. Autozero Concentration = | 5.00 |
| | Inject Position ECD_1.AcqOn Concentration = | 5.00 |
| 28.000 | Concentration = | 30.00 |
| 28.00 | ECD_1.AcqOff Concentration = | 30.00 |
| | Wait | Ready |

D) The required chemicals are (a) deionized water with a specific resistivity of 17.8 megohm-cm or greater, (b) 1,2,4-trichlorobenzene, HPLC grade, (c) sodium sulfite, reagent grade and (d) 0.1 wt % solution of sodium sulfite in water.
E) For standardization quality control, a standard solution “B” is prepared as follows: A concentrated bromide standard solution (1,000 μg/mL) is prepared by weighing 0.1287 g of sodium bromide into a 100-mL volumetric flask, diluting to volume with deionized water and mixing well. This is standard solution “A”. The bromide calibration standard solution “B” is prepared by pipetting 100 μL of the concentrated bromide standard solution into a 100-mL volumetric flask which is then filed to volume with deionized water and mixed well. This provides a standard solution “B” of 1 μg/mL as bromide. Two aliquots of the latter bromide calibration standard solution are loaded into individual polyseal autosampler vials for duplicate analysis.
F) In conducting the analyses it is recommended to prepare duplicate samples for both the occluded bromine/bromide determination and for the uncorrected ionic bromine (bromide) determination, so that a total of four sample preparations is used for each sample that is analyzed. The detailed analytical procedure involves the following: (a) Approximately 0.030 g of the sample is weighed into a 40-mL amber glass EPA vial. (b) 20 mL of 1,2,4-trichlorobenzene is added to the vial using a volumetric pipet, the vial is capped tightly with the septum cap and the vial is shaken slightly and sonicated to wet the sample. (c) A blank is prepared as above containing only 20 mL of 1,2,4-trichlorobenzene. (d) The vials are placed in a heating block at approximately 95 C for 10 minutes with occasional shaking until the sample has dissolved. (e) For determining ionic bromine (bromide), the vial is removed from the bath and exactly 5 mL of deionized water is immediately added through the septum cap by means of a syringe. The vial remains sealed. (f) For determining total occluded bromine and bromide the vial is removed from the bath and exactly 5 mL of sodium sulfite solution in deionized water is immediately added through the septum cap by means of a syringe. The vial remains sealed. (g) Each vial is shaken on a shaker for 20 minutes. (h) Using a disposable pipet, the upper aqueous layer is removed and filtered through a GHP Polypro syringe filter. (i) 25 μL of the filtered sample is injected into the ion chromatograph and analyzed using the above operating conditions.
G) The calculations used are as follows:
   a) This method uses the response factor calculated from duplicate injections of the individual standard solution “B”. The response factor is calculated using the equation:

$$RF = \frac{Avg . Peak Area ( 2 injections )}{Standard Concentration ( µg \text{/} mL )}$$

b) The area of the bromide peak for each sample run is corrected for the area of the bromide peak in the blank in accordance with the expression:

A_{S}−A_{b}=A
where: A_{S} is the area of the sample peak; A_{b} is the area of the blank peak; A is the corrected area of the sample peak.
c) The corrected bromide area for each sample preparation is used to determine the total concentration of occluded bromine and bromide in the sample using the expression:

$$ppm Br = \frac{A \times V}{RF \times W}$$

where A is the corrected area of the sample, RF is the response factor for bromide, W is the amount of sample expressed in grams (approximately 0.03 g) and V is the total volume of the aqueous solution (5 mL).
d) The levels of occluded bromine and ionic bromine (bromide) are calculated from the duplicate average results for total occluded bromine and bromide (sulfite treated) and for uncorrected ionic bromine (no sulfite) using the expression:

ppm occluded bromine=2×(ppm of total occluded bromine and bromide)−(ppm uncorrected bromine).

An example of a preferred process of this invention in which the solids formed during the bromination are broken up by grinding, milling, or sonication is a process in which the Lewis acid bromination catalyst is charged to the reactor as a mixture of aluminum chloride in bromine, or more preferably as a solution of aluminum bromide in bromine, and in which the aromatic compound to be brominated is 1,2-diphenylethane, and in which the brominated aromatic flame retardant to be prepared in the process is a decabromodiphenylethane product, where the bromination is conducted at a temperature in the range of about 50 to about 55° C. Such process is capable of producing a decabromodiphenylethane product containing over 99.5 GC area percent of decabromodiphenylethane and having a nonabromodiphenylethane content of 0.5 GC area percent or less, preferably 0.3 GC area percent or less, and more preferably, 0.1 GC area percent or less.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Each and every patent or publication referred to in any portion of this specification is incorporated in toto into this disclosure by reference, as if fully set forth herein.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

1. A process of preparing brominated aromatic flame retardant having a reduced content of occluded free bromine, which process comprises subdividing during bromination of an aromatic compound, solid particles that form in a bromination reaction mixture in which an excess of liquid bromine and a Lewis acid bromination catalyst are present, said subdividing taking place within the confines of said reaction mixture.

2. A process as in claim 1 wherein the subdividing is accomplished by grinding or milling.

3. A process as in claim 1 wherein the subdividing is accomplished by sonication.

4. A process as in claim 1 wherein the Lewis acid bromination catalyst is an aluminum-based Lewis acid catalyst.

5. A process as in claim 1 wherein said aromatic compound is 1,2-diphenylethane.

6. A process as in claim 1 wherein said aromatic compound is diphenyl oxide.

7. A process as in claim 1 wherein the Lewis acid bromination catalyst is an aluminum-based Lewis acid catalyst, wherein said aromatic compound is 1,2-diphenylethane, and wherein the brominated aromatic flame retardant being prepared in said process is a decabromodiphenylethane product.

8. A process as in claim 1 wherein the Lewis acid bromination catalyst is an aluminum-based Lewis acid catalyst, wherein said aromatic compound is diphenyloxide, and wherein the brominated aromatic flame retardant being prepared in said process is a decabromodiphenyl oxide product.

9. A process as in claim 2 wherein the Lewis acid bromination catalyst is an aluminum-based Lewis acid catalyst, wherein said aromatic compound is 1,2-diphenylethane, and wherein the brominated aromatic flame retardant being prepared in said process is a decabromodiphenylethane product.

10. A process as in claim 2 wherein the Lewis acid bromination catalyst is an aluminum-based Lewis acid catalyst, wherein said aromatic compound is 1,2-diphenylethane, and wherein the brominated aromatic flame retardant being prepared in said process is a decabromodiphenylethane product.

11. A process as in claim 2 wherein the Lewis acid bromination catalyst is an aluminum-based Lewis acid catalyst, wherein said aromatic compound is 1,2-diphenylethane, and wherein the brominated aromatic flame retardant being prepared in said process is a decabromodiphenylethane product.

12. A process as in claim 3 wherein the Lewis acid bromination catalyst is an aluminum-based Lewis acid catalyst, wherein said aromatic compound is 1,2-diphenylethane, and wherein the brominated aromatic flame retardant being prepared in said process is a decabromodiphenylethane product.
